(19) United States
(12) Patent Application Publication
(10) Pub. No.: US 2007/0116583 A1
(43) Pub. Date: May 24, 2007

(54) FUEL SUPPLY PUMP

(76) Inventor: Nobuo Aoki, HIGASHIMATSUYAMA-SHI, SAITAMA (JP)

Correspondence Address: ARTHUR G. SCHAIER;CAMODY & TORRANCE LLP, 50 LEAVENWORTH STREET, P.O. BOX 1110, WATERBURY, CT, 06721-1110, US

(21) Appl. No.: 10/581,161

(22) PCT Filed: Jan. 7, 2005
(86) PCT No.: PCT/JP05/00099

§ 371 Date: May 31, 2006

(30) Foreign Application Priority Data
Jan. 14, 2004 (JP) 2004-006200

(51) Int. Cl. A61M 1/00

(52) U.S. Cl. 417/437

(57) ABSTRACT

The present invention provides a fuel supply pump which is suitably used in a booster-type accumulator fuel injection device and a tappet structural body which is suitable for the fuel supply pump. For this end, in a fuel supply pump which includes a plurality of plunger barrels, plungers and tappet structural bodies in the inside of a pump housing, a plurality of accommodating portions for arranging the plurality of plunger barrel in parallel to each other is formed in the inside of the pump housing and an inter-cylinder connecting portion which allows lubricant or lubricating fuel to pass therethrough is provided between the plurality of accommodating portions.

### TECHNICAL FIELD

The present invention relates to a fuel supply pump, and more particularly to a fuel supply pump which is suitably applicable to a booster-type accumulator fuel injection device.

### BACKGROUND ART

Conventionally, in a diesel engine or the like, to inject a high-pressure fuel efficiently, various accumulator fuel injection devices which use an accumulator (common rail) have been proposed.

As a fuel supply pump which is used to such an accumulator fuel injection device, there has been adopted a fuel supply pump which includes, in the inside of a pump housing thereof, a cam which is rotatably integrated with a cam shaft which is rotated by driving an engine, a plunger which is elevated due to the rotation of the cam, a tappet structural body which transmits the rotation of the cam to the plunger as a rising force, and a plunger barrel in which the plunger is arranged.

In such a fuel supply pump, as shown in FIG. 18, a plunger barrel 407 is arranged in accommodating portion in the inside of a pump housing 402 and a plunger 410 is partially inserted into the inside of the plunger barrel 407 in a vertically movable manner. Further, the plunger 410 is elevated due to the cam 404 and the tappet structural body 406 and is lowered due to a return spring 411, wherein due to the repetition of elevation and lowering of the plunger 410, the fuel is pressurized and is supplied to an accumulator (see, Patent Document 1, for example).

Further, usually, to supply a large quantity of the high-pressure fuel to the accumulator, these plungers, the tappet structural bodies and the like are arranged in plural numbers and the fuel is subject to pressurizing processing in the respective plungers.

[Patent Document 1] JP-A-2001-317430 (FIG. 1)

### DISCLOSURE OF THE INVENTION

### PROBLEMS TO BE SOLVED BY THE INVENTION

However, in the fuel supply pump disclosed in Patent Document 1, the sufficient pressurizing processing of a large flow rate of fuel by rotating the fuel supply pump at a high speed is not taken into consideration at all. Accordingly, all of a plurality of accommodating portions in which the plungers and the tappet structural bodies are accommodated are independently constituted. Accordingly, when the pump is rotated at a high speed, a lubricant remains or stays in a spring holding chamber which constitutes a portion of the accommodating portion and hence, the pressure inside a spring holding chamber is elevated thus impeding the operation of the plunger. Accordingly, there has been a drawback that a large quantity of fuel cannot be sufficiently subject to pressurizing processing.

In view of the above circumstances, inventors of the present invention have made extensive studies and have found that by providing a predetermined inter-cylinder connecting portion in the inside of a pump housing, the reciprocation of lubricant or lubrication fuel is carried out smoothly and hence, even when a pump is rotated at a high speed, it is possible to prevent the excessive elevation of pressure in the inside of a spring holding chamber.

That is, it is an object of the present invention to provide a fuel supply pump which, even when a fuel supply pump is rotated at a high speed to cope with a booster-type accumulator fuel injection device, can perform the sufficient pressurizing processing of fuel while preventing lubricant or lubrication fuel from impeding the operation of plungers.

### MEANS FOR SOLVING THE PROBLEMS

According to the present invention, there is provided a fuel supply pump which includes a plurality of plunger barrels, plungers and tappet structural bodies in the inside of a pump housing, wherein a plurality of accommodating portions for arranging a plurality of plunger barrels in parallel are formed in the inside of the pump housing, and an inter-cylinder connecting portion which allows lubricant or lubrication fuel to pass therethrough between the plurality of accommodating portions whereby it is possible to overcome the above-mentioned drawbacks.

That is, by providing the predetermined inter-cylinder connecting portion in the inside of the pump housing, a moving space for the lubricant or the lubrication fuel is formed and hence, it is possible to prevent the excessive elevation of the pressure in the inside of the spring holding chamber. That is, in the fuel supply pump which includes the plurality of plunger barrels, the plungers and the like, usually, the plungers are configured to repeat the vertical reciprocating motion alternatively. Here, when one plunger is elevated and a volume of the spring holding chamber corresponding to the plunger is decreased, another plunger is lowered and a volume of the spring holding chamber corresponding to the plunger is increased. Accordingly, even when a volume of one spring holding chamber is decreased, the lubricant or the lubrication fuel can move to another spring holding chamber from one spring holding chamber by way of the inter-cylinder connecting portion and hence, it is possible to prevent the elevation of the pressure.

Accordingly, a possibility that the lubricant or the lubrication fuel impedes the high-speed driving of the plungers can be reduced.

Further, in constituting the fuel supply pump of the present invention, it is preferable to arrange the inter-cylinder connecting portion at a position higher than elevated positions of the tappet structural bodies.

Further, in constituting the fuel supply pump of the present invention, it is preferable to arrange the inter-cylinder connecting portion substantially perpendicular to or in an inclined manner with respect to the arranging direction of the plurality of plunger barrels.

Further, in constituting the fuel supply pump of the present invention, it is preferable to set a cross-sectional area of the inter-cylinder connecting portion to a value which falls within a range of 10 to 350 mm².

Further, in constituting the fuel supply pump of the present invention, it is preferable to provide a valve portion in a middle portion of the inter-cylinder connecting portion.

Further, in constituting the fuel supply pump of the present invention, it is preferable to form communicating portions which allow the lubricant or the lubricating fuel to pass through the tappet structural bodies in the tappet structural bodies.

Further, in constituting the fuel supply pump of the present invention, the fuel supply pump is preferably applicable to a booster-type accumulator fuel injection device which pressurizes a fuel having a flow rate per unit time of 500 to 1500 litter/hour to a value which exceeds 50 MPa or more.

### BRIEF DESCRIPTION OF THE DRAWINGS

FIG. 1 is a side view with a part broken away of a fuel supply pump of the present invention.

FIG. 2 is a cross-sectional view of a fuel supply pump of the present invention.

FIG. 3(a) to FIG. 3(b) are respectively a perspective view and a cross-sectional view of a pump housing.

FIG. 4(a) to FIG. 4(b) are respectively views for explaining an arrangement method of an inter-cylinder connecting portion (first case).

FIG. 5(a) to FIG. 5(b) are respectively views for explaining an arrangement method of an inter-cylinder connecting portion (second case).

FIG. 6(a) to FIG. 6(b) are respectively views for explaining an arrangement method of an inter-cylinder connecting portion (third case).

FIG. 7 is a view for explaining a valve portion formed in an inter-cylinder connecting portion.

FIG. 8 is a view for explaining a pressure change in the inside of a spring holding chamber.

FIG. 9(a) to FIG. 9(c) are respectively a perspective view, a plan view and a cross-sectional view of another spring seat.

FIG. 10(a) to FIG. 10(b) are respectively views for explaining a tappet structural body (first case).

FIG. 11(a) to FIG. 11(c) are respectively views for explaining a tappet structural body (second case).

FIG. 12(a) to FIG. 12(c) are respectively views for explaining a roller body.

FIG. 13 is a view for explaining a roller in the tappet structural body.

FIG. 14 is a view for explaining a system of a booster-type accumulator fuel injection device.

FIG. 15 is a view for explaining the structure of a booster-type accumulator fuel injection device.

FIG. 16 is a view which conceptually shows a fuel boosting method in the booster-type accumulator fuel injection device.

FIG. 17 is a view for explaining an injection timing chart of high-pressure fuel.

FIG. 18 is a view for explaining a conventional fuel supply pump.

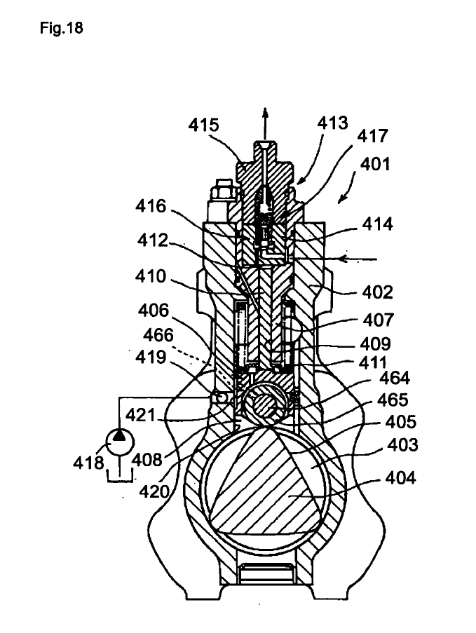

### BEST MODE FOR CARRYING OUT THE INVENTION

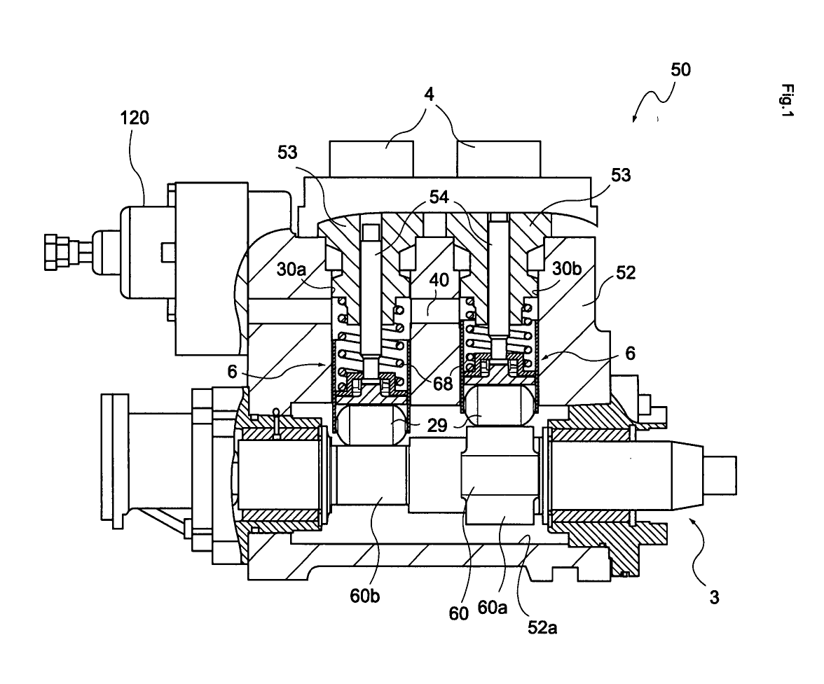

This embodiment, as shown in FIG. 1, is directed to a fuel supply pump 50 which includes a plurality of plunger barrels 53, plungers 54 and tappet structural bodies 6 in the inside of a pump housing 52. Here, in the inside of the pump housing 52, a plurality of accommodating portions 30a, 30b are formed for arranging the plurality of plunger barrels 53 parallel to each other and an inter-cylinder connecting portion 40 which allows lubricant or lubricating fuel to pass therethrough is provided between the plurality of accommodating portions 30a, 30b.

Hereinafter, the fuel supply pump 50 is explained in detail with respect to respective constitutional features thereof by taking a fuel supply pump which includes two sets of plunger barrels 53 and accommodating portions 30 as an example. However, this embodiment describes merely one aspect of the present invention and dose not limit the present invention, and the embodiment can be modified arbitrarily within the scope of the present invention.

1. Basic Configuration of Fuel Supply Pump

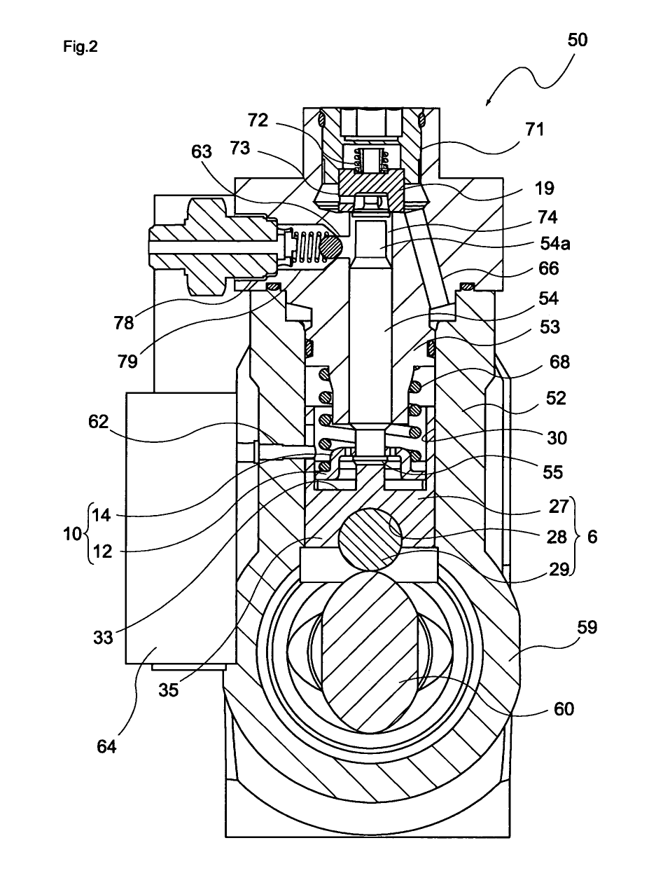

Although the basic configuration of the fuel supply pump is not particularly limited, for example, it is preferable to adopt the structure of the fuel supply pump 50 shown in FIG. 1 and FIG. 2. That is, the fuel supply pump 50 preferably includes, for example, the pump housing 52, the plunger barrels (cylinders) 53, the plungers 54, a spring seat 10, the tappet structural bodies 6 and cams 60.

Further, in the inside of each plunger barrel 53 which is housed in the pump housing 52, a fuel compression chamber 74 is formed, wherein the plunger 54 reciprocates in the fuel compression chamber 74 in response to the rotary movement of the cam 60 so as to pressurize the fuel introduced into the fuel compression chamber 74. Accordingly, in the fuel compression chamber 74, it is possible to efficiently pressurize the fuel which is forcibly fed from a feed pump to form a high-pressurized fuel using the plunger 54.

Here, in the fuel supply pump 50 of this embodiment, for example, although two sets of the plunger barrels 53 and plungers 54 are provided in the inside of the pump housing 52, for processing a large quantity of fuel at a high-pressure, it may be also preferable to increase the number of sets of the plunger barrels 53 and plungers 54 to two or more.

(1) Pump Housing

As exemplified in FIG. 2, the pump housing 52 is a housing which accommodates the plunger barrels 53, the plungers 54, the tappet structural bodies 6 and the cams 60.

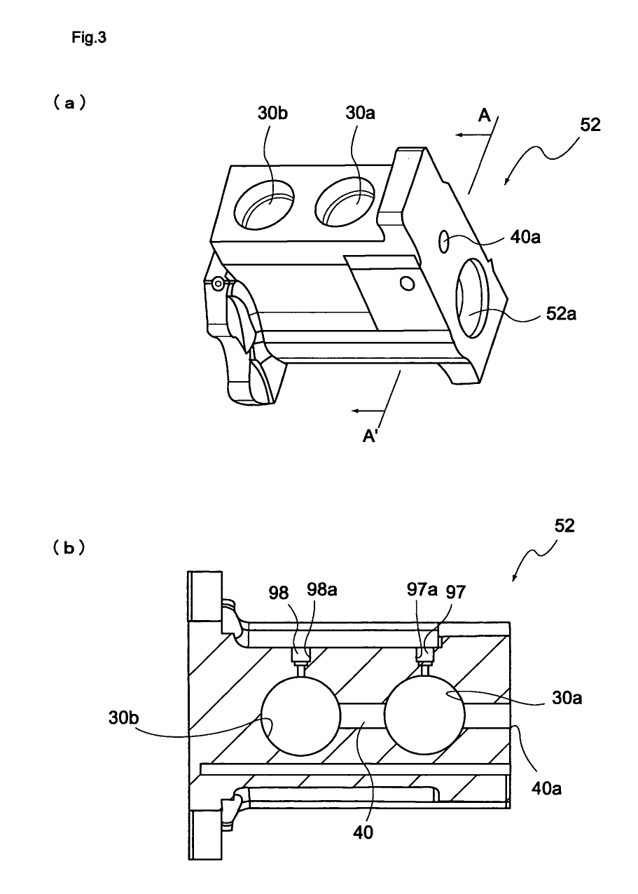

Accordingly, as shown in FIG. 3(a) and FIG. 3(b), it is preferable that the pump housing 52 includes a shaft insertion hole 92a which is opened in the lateral direction and columnar spaces 30a, 30b which are opened in the vertical direction as a plurality of accommodating portions 30 for arranging the plurality of plunger barrels 53 in parallel to each other. Further, spring holding chambers may preferably be formed by arranging the plunger barrels, the tappet structural bodies and springs described later which impart a lowering force to the tappet structural bodies in the columnar spaces. Further, in the fuel supply pump of the present embodiment, an inter-cylinder connecting portion 40 which allows the lubricant or the lubricating fuel to pass therethrough is provided between the columnar spaces 30a, 30b.

Further, as shown in FIG. 3(b), through holes 97, 98 which open in the side surface direction of the columnar spaces 30a, 30b may also be preferably formed in the pump housing 52. That is, distal end portions of guide pins (not shown in the drawings) which prevent the tappet structural bodies from being rotated in the circumferential direction and guide vertical movement positions of the tappet structural bodies are pushed into the through holes 97, 98 for ensuring the positioning accuracy of the guide pins. Further, it is preferable that hole portions 97a, 98a are formed of a threaded portion with which the guide pin is threadedly engaged and the distal end portions of the guide pins are pushed into the holes 97a, 98a by threaded engagement.

Here, in this embodiment, the explanation is made by taking the pump housing which includes two columnar spaces which form a plurality of accommodating portions as an example. However, this embodiment is not limited to such a constitution and there may be a case that the pump housing includes three or more columnar spaces.

(2) Inter-cylinder Connecting Portion

(2)-1 Summary

The fuel supply pump of this embodiment is, as shown in FIG. 1 and FIG. 3(b), characterized in that the inter-cylinder connecting portion 40 which allows lubricant or lubricating fuel to pass therethrough is provided between the columnar spaces 30a, 30b constituting a plurality of accommodating portions for arranging the plunger barrels 53 in parallel to each other. That is, by arranging the predetermined inter-cylinder connecting portion 40 between the columnar spaces 30a, 30b which are formed in the pump housing 52, the lubricant or the lubricating fuel is reciprocated between the plurality of accommodating portions 30a, 30b by way of the inter-cylinder connecting portion 40 and hence, it is possible to prevent the pressure of the spring holding chamber constituting a part of the columnar space 30a, 30b from rising excessively.

To be more specific, in the fuel supply pump including the plurality of plungers and the like, usually, the plungers are constituted so as to respectively repeat a vertical reciprocating motion alternately. Further, when one plunger is elevated and a volume of the spring holding chamber corresponding to the plunger is decreased, another plunger is lowered and a volume of another spring holding chamber corresponding to another plunger is increased. Here, with the provision of the inter-cylinder connecting portion, even when the volume of one spring holding chamber is decreased, the lubricant or the like which exists in the inside of the spring holding chamber is allowed to move into another spring holding chamber by way of the inter-cylinder connecting portion. Accordingly, it is possible to prevent the lubricant or the like from remaining in the inside of the spring holding chamber.

Accordingly, with the provision of the inter-cylinder connecting portion in the fuel supply pump, it is possible to prevent the lubricant or the like in the inside of the spring holding chamber from impeding the high-speed driving of the cam and the plunger.

(2)-2 Arrangement 1

Further, as shown in FIG. 1, the inter-cylinder connecting portion may preferably be, in the accommodating portion 30, arranged to a higher position than an elevated position of the tappet structural body 6.

This is because that such a provision can prevent a phenomenon that when the tappet structural body and the plunger are elevated due to the rotation of the cam, the inter-cylinder connecting portion is clogged by the tappet structural body. Accordingly, a possibility that the lubricant or the like remains in the inside of the spring holding chamber and impedes the high-speed driving of the plunger is decreased.

Here, in FIG. 1, although the right-side tappet structural body 6 is arranged at the highest elevated position thereof, the inter-cylinder connecting portion 40 is arranged to a higher position than the highest elevated position of the tappet structural body 6.

(2)-2 Arrangement 2

Further, as shown in FIG. 1 and FIG. 3(b), the inter-cylinder connecting portion may preferably be arranged substantially perpendicular to the arranging direction of the plunger barrels between the columnar spaces 30a, 30b which constitute the accommodating portion 30 of the pump housing 52. This is because that, due to such a constitution, respective heights of the inter-cylinder connecting portion at positions in the columnar spaces 30a, 30b become equal to each other. Accordingly, conditions for elevating and lowering the pressure in the inside of the respective spring holding chambers can be made equal thus preventing the generation of irregularities between these conditions.

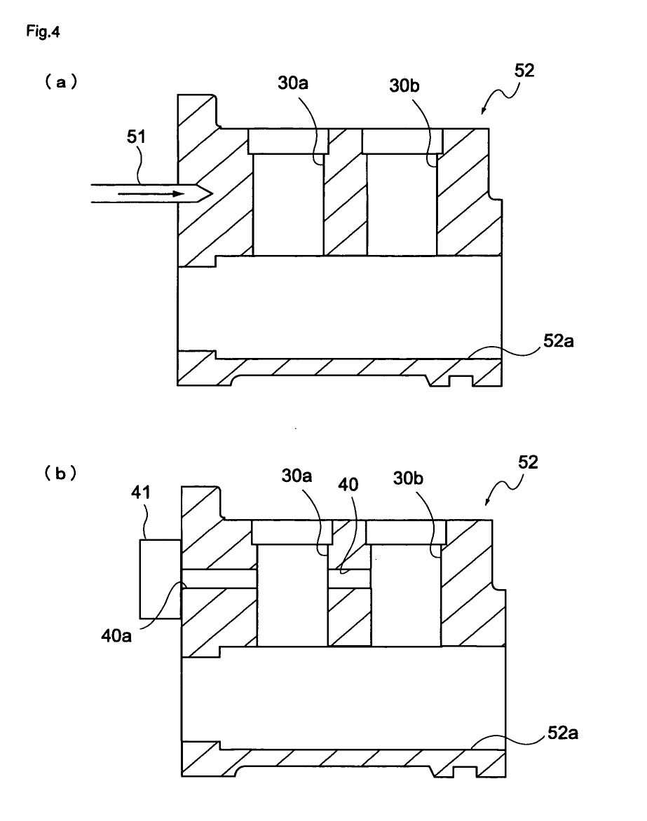

Further, when the inter-cylinder connecting portion is arranged as described above, as shown in FIG. 4(a) to FIG. 4(b), the inter-cylinder connecting portion can be formed using a drill 51 or the like from a side-surface side of the pump housing 52. Further, an inlet portion 40a which is formed at the side-surface side of the inter-cylinder connecting portion 40 may preferably be closed by a sealing member 41 thus sealing the inter-cylinder connecting chamber. Due to such a constitution, the predetermined inter-cylinder connecting portion can be formed easily and the leaking of the lubricant or the like can be prevented.

(2)-3 Arrangement 3

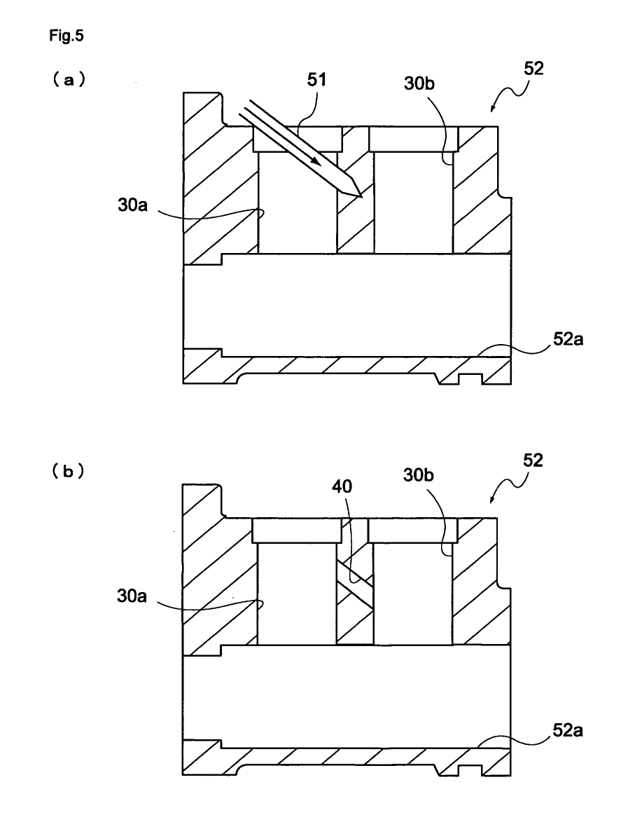

Further, as shown in FIG. 5(a) to FIG. 5(b), the inter-cylinder connecting portion 40 may preferably be formed in an inclined manner with respect to the reciprocating direction of the plunger 54. That is, as shown in FIG. 5(a), at the time of manufacturing the inter-cylinder connecting portion 40, the inter-cylinder connecting portion 40 may be formed in the oblique direction from above one columnar space 30a using the drill 51 and the like and hence, it is unnecessary to form holes or the like other than a hole or the like which functions as the inter-cylinder connecting portion. Accordingly, when the tappet structural body, the plunger barrel and the like are mounted in the pump housing, the inter-cylinder connecting portion is surely sealed and hence, the leaking of the lubricant or the like can be prevented.

(2)-4 Arrangement 4

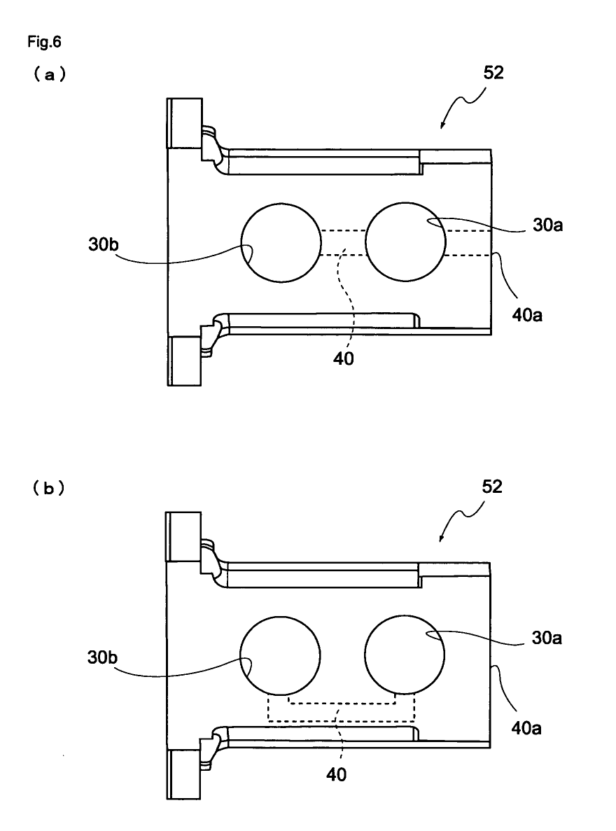

Further, as shown in FIG. 6(a), the inter-cylinder connecting portion may preferably be formed straightly so as to communicably connect columnar space 30a and another columnar space 30b with the shortest distance.

This is because that the inter-cylinder connecting portion which is formed as described above can allow the lubricant and the like to pass therethrough more smoothly compared with a case as shown in FIG. 6(b) in which the inter-cylinder connecting portion is formed in a roundabout manner between one columnar space 30a and another columnar space 30b. Further, in the case shown in FIG. 6(b), the inter-cylinder connecting portion may be arranged in the vicinity of a side wall of the pump housing thus lowering a mechanical strength of the pump housing.

(2)-5 Cross-sectional Area

Further, a sectional area of the inter-cylinder connecting portion may preferably be set to a value which falls within a range of 10 to 350 mm².

This is because that, when the cross-sectional area of the inter-cylinder connecting portion becomes less than 10 mm², the lubricant or the like hardly reciprocates between the plurality of spring holding chambers and hence, the pressure in the inside of the spring holding chamber may be increased. On the other hand, when the sectional area of the inter-cylinder connecting portion exceeds 350 mm², there may be a case that a mechanical strength of the pump housing is decreased.

Accordingly, it is more preferable to set the cross-sectional area of the inter-cylinder connecting portion to a value which falls within a range of 30 to 300 mm², and it is still more preferable to set the cross-sectional area of the inter-cylinder connecting portion to a value which falls within a range of 50 to 250 mm².

Here, the cross-sectional area of the inter-cylinder connecting portion indicates, when the plurality of inter-cylinder connecting portions is formed in the pump housing, a total area of the respective cross-sectional areas of the respective inter-cylinder connecting portions.

(2)-6 The Number

Further, the number of inter-cylinder connecting portions is not particularly limited and one inter-cylinder connecting portion having a comparatively large area can be formed in the pump housing or a plurality of inter-cylinder connecting portions having a comparatively small area can be formed in the pump housing. Further, a plurality of inter-cylinder connecting portions which have different cross-sectional areas may be formed.

However, one inter-cylinder connecting portion having the comparatively large area may preferably be formed in the pump housing. This is because that the inter-cylinder connecting portion having the comparatively large area allows the high-pressure lubricant or the like to reciprocate smoothly and, also can reduce the occurrence of clogging.

(2)-7 Valve Portion

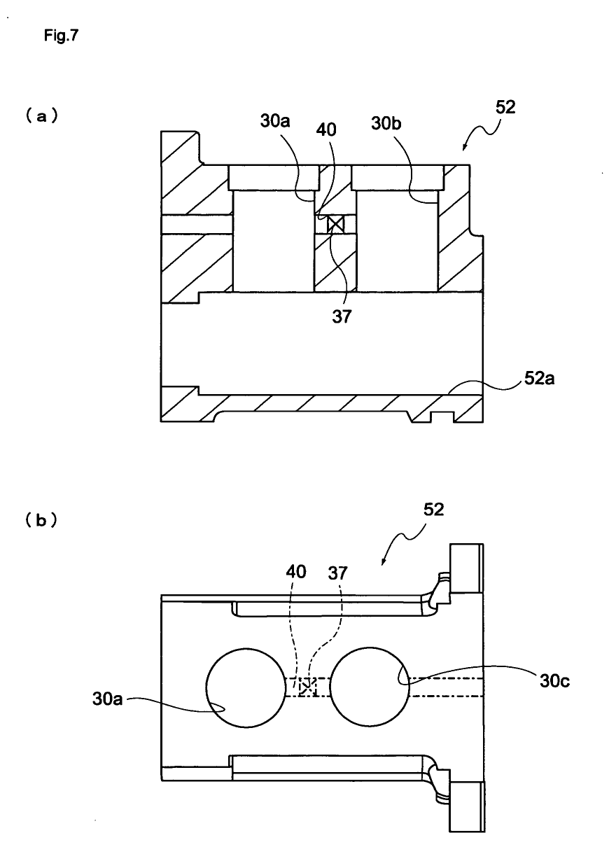

Further, as shown in FIG. 7(a) and FIG. 7(b), a valve portion 37 may preferably be arranged in a midst portion of the inter-cylinder connecting portion 40.

This is because that, with the provision of the valve portion, for example, only when the pressure in the inside of the spring holding chamber on a side at which the plunger is elevated exceeds the predetermined value, the valve portion is opened so as to allow the lubricant or the like to pass therethrough. Accordingly, it is possible to easily adjust the pressure in the inside of the spring holding chamber. Further, due to such an adjustment, the pressures in the inside of the respective spring holding chambers are made uniform thus effectively preventing the occurrence of irregularities with respect to a flow rate of the fuel supplied from the pump.

(2)-8 Peaks of Pressure

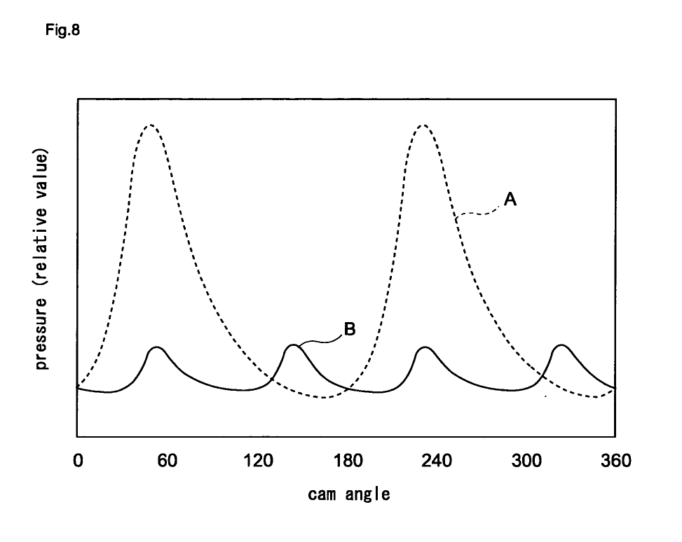

Here, by reference to FIG. 8, a change in pressure in the inside of the spring holding chamber is explained hereinafter when the cam is rotated by 360°. In FIG. 8, a broken line A indicates, in the fuel supply pump which is not provided with the inter-cylinder connecting portion according to the present invention, a profile of a change in pressure in the inside of the predetermined spring holding chamber along with the rotation of the cam. Further, in FIG. 8, a solid line B indicates, in the fuel supply pump which is provided with the inter-cylinder connecting portion (effective cross-sectional area 200 mm²) according to the present invention, a profile of a change in pressure in the inside of the predetermined spring holding chamber accompanied with the rotation of the cam. Further, the pressure (relative value) is taken on an axis of abscissas and a cam angle (degree) is taken on an axis of ordinates. Here, each fuel supply pump includes two spring holding chambers and uses the elliptical cams.

As indicated by the broken line A, in the profile of the change in pressure in the inside of the spring holding chamber of the fuel supply pump which is not provided with the inter-cylinder connecting portion, two peaks which correspond to the shape of the cam exist during the rotation of the cam by 360°. That is, at the respective peaks in pressure, the plunger is elevated by the cam so that a volume of the spring holding chamber is decreased whereby the pressure in the inside of the spring holding chamber is increased. Further, the pressures at respective peaks assume relatively high values.

On the other hand, as indicated by the solid line B, in the profile of the change in pressure in the inside of the spring holding chamber of the fuel supply pump which is provided with the inter-cylinder connecting portion according to the present invention, four peaks exist during the rotation of the cam by 360°. This is because that since a cam which elevates two plungers is arranged with their phases displaced from each other by 90°, one plunger and another plunger are respectively elevated twice and hence, during the rotation of the cams by 360°, these plunger are elevated 4 times in total. That is, when the cam is rotated by 60°, the plunger corresponding to the spring holding chamber on a side where the pressure is measured is lowered, while the plunger corresponding to the spring holding chamber on another side is elevated and, at the same time, a volume of the spring holding chamber on another side is decreased. Accordingly, the lubricant or the like moves to the spring holding chamber on the side where the volume is increased and the pressure is measured. Thus, a first peak appears.

Next, when the cam is rotated by 150°, the plunger corresponding to the spring holding chamber of a side where the pressure is measured is elevated and a volume of the spring holding chamber is decreased and hence, the pressure in the inside of the spring holding chamber is elevated. In this manner, the second peak appears. Here, the plunger on another side is lowered and the volume of the spring holding chamber on another side is increased and hence, the lubricant or the like is moved to the spring holding chamber on another side.

Thereafter, these operations are repeated and hence, in response to the elevation of the respective plungers, pressure peaks appear in the inside of the spring holding chambers.

Here, the pressures at respective peaks are, since the lubricant or the like in the spring holding chamber is smoothly moved to the side on which the volume is increased, lowered to approximately 15% compared to a case in which the above-mentioned inter-cylinder connecting portion is not provided.

Accordingly, it is understood that the provision of the inter-cylinder connecting portion which allows the respective spring holding chambers to be communicated with each other is effective for lowering the maximum pressure in the inside of the spring holding chamber.

(3) Plunger Barrel (Cylinder)

The plunger barrels 53 are, as illustrated in FIG. 1 and FIG. 2, housings for supporting the plungers 54 and are elements which constitute portions of the fuel compression chambers (pump chambers) 74 for pressurizing a large quantity of fuel to a high pressure using the plungers 54. Further, the plunger barrel 53 may preferably be mounted on upper opening portions of columnar spaces 30a, 30b of the pump housing 52 for facilitating the assembling.

Here, when the type of fuel supply pump on which the plunger barrels are mounted is either an in-line type or a radial type, the configuration of the plunger barrels may suitably be changed corresponding to the respective types.

(4) Plunger

The plungers 54 are, as illustrated in FIG. 1 and FIG. 2, main elements for pressurizing the fuel in the fuel compression chambers 74 formed in the inside of the plunger barrels 53 to a high pressure. Accordingly, the plungers 54 may preferably be elevatably arranged in the inside of the plunger barrels 53 which are respectively mounted in the columnar spaces 30a, 30b of the pump housing 52.

Here, to enable the pressurizing processing of the large quantity of fuel by driving the plungers at a high speed, it is preferable to set a rotational speed of the pump to a value which falls within a range of 1500 to 4000 rpm and, at the same time, it is preferable to set the rotational speed of the pump to a value which falls within a range of 1 to 5 times as large as a rotational speed of the engine by taking a gear ratio into consideration.

(5) Fuel Compression Chamber

The fuel compression chamber 74 is, as shown in FIG. 2, a small chamber which is formed in the inside of the plunger barrel 53 together with the plunger 54. Accordingly, in such a fuel compression chamber 74, by driving the plunger 54 at a high speed, it is possible to efficiently pressurize a large quantity of the fuel which quantitatively flows in the fuel compression chamber 74 by way of the fuel supply valve 73. Here, as will be described later, according to the fuel supply pump of the present invention, it is possible to obtain an advantage that even when the plunger 54 is driven at a high speed, the lubricant or the lubrication fuel in the inside of the spring holding chamber does not impede the high-speed operation of the plunger 54.

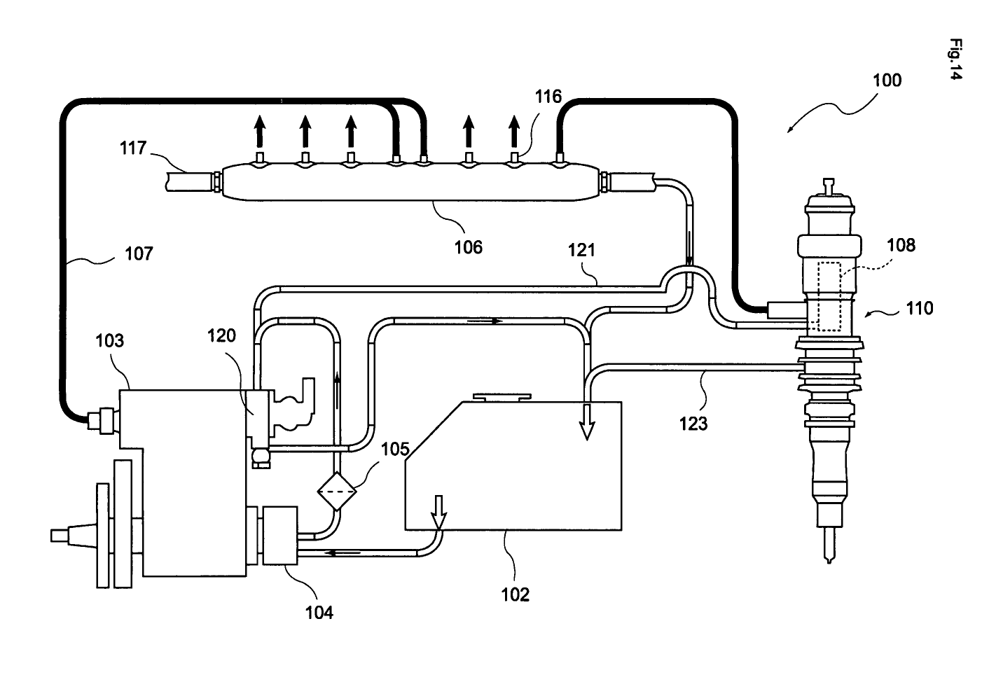

On the other hand, after the pressurizing using the plunger 54 is finished, the pressurized fuel is supplied to a common rail 106 shown in FIG. 14 by way of a fuel discharge valve 79.

(6) Spring Seat

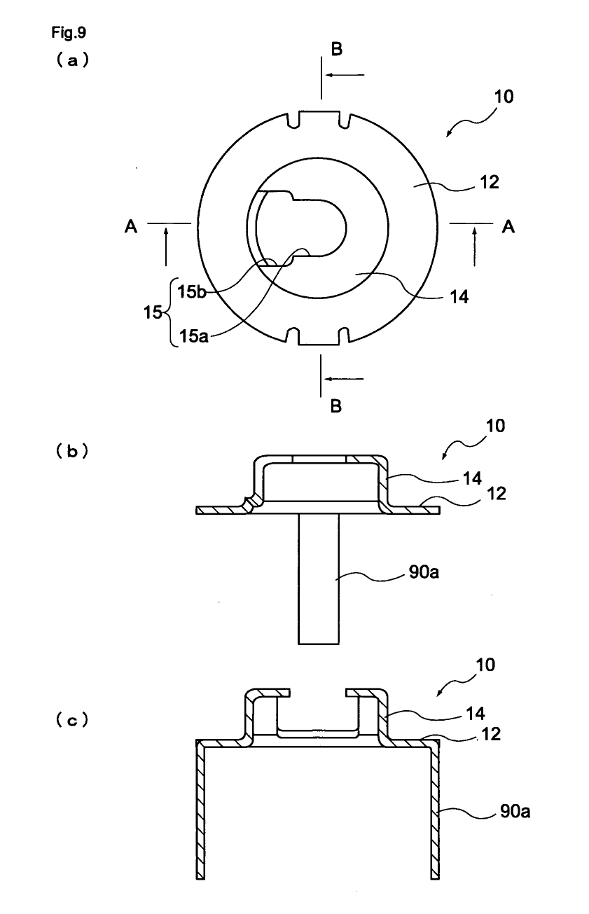

The spring seat 10 is an element for holding a return spring which is used for pulling down the plunger of the fuel supply pump. With respect to such a spring seat 10, as shown in FIG. 9(a) to FIG. 9(c), it is preferable that a portion of a peripheral portion of the spring seat 10 is extended in the direction toward an end portion of the roller, and an extending portion is constituted as a restricting means 90a which restricts the movements of the roller in the tappet structural body in the rotary axis direction. The reason is that due to such a constitution, even when the pump is rotated at a high speed, it is possible to prevent an inner peripheral surface of the pump housing from being damaged by an end portion of the roller.

Here, by constituting the spring seat in the above-mentioned manner, it is possible to allow an insertion hole which is formed in a tappet body portion and in which the restricting means is inserted to function also as a communication hole for the lubricant or the like.

(7) Tappet Structural Body

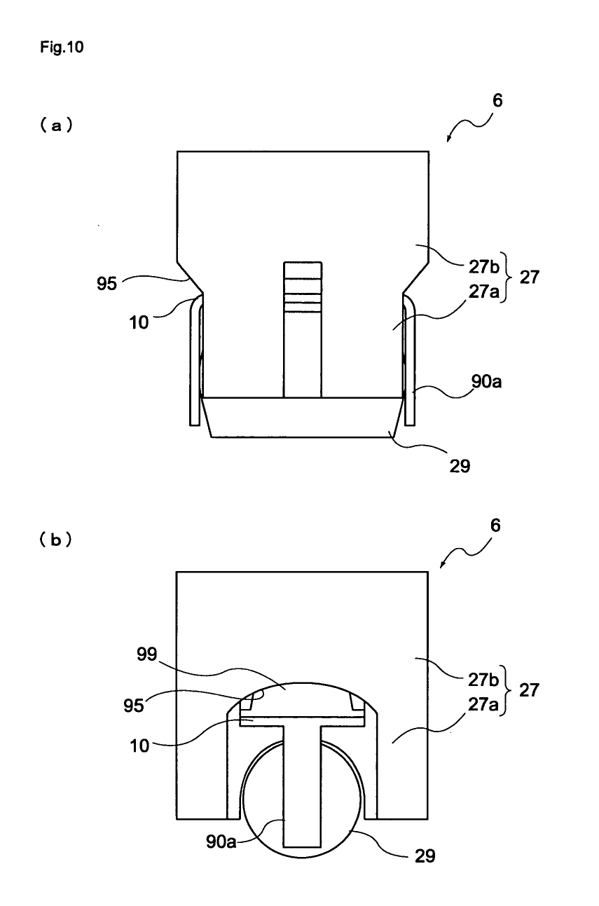

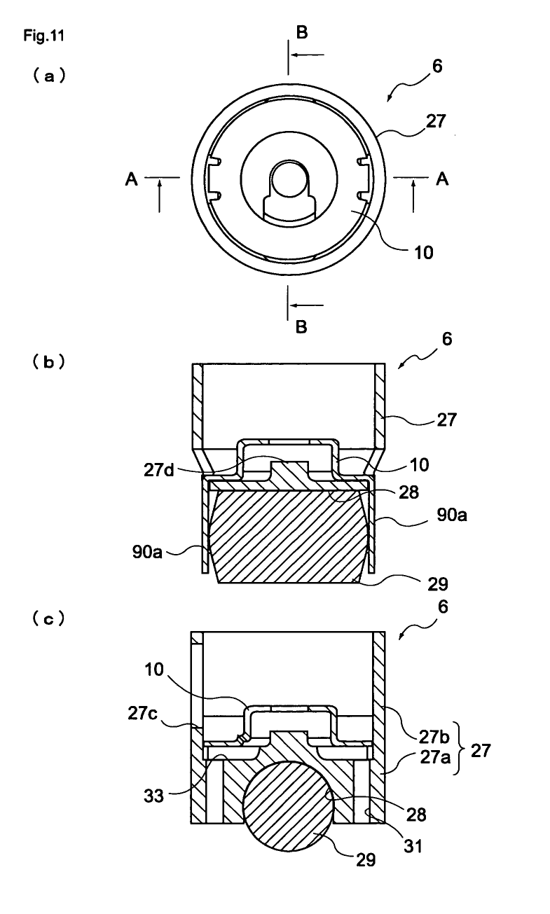

The tappet structural body 6 is, as shown in FIG. 10(a) to FIG. 10(b) and FIG. 11(a) to FIG. 11(c), basically constituted of a tappet body portion 27 which is formed of a body portion 27a made of a block body and a cylindrical slide portion 27b which is extended from the body portion 27a and a roller 29. The tappet structural body 6 may preferably be constituted such that the tappet structural body 6 is elevated due to the rotational movement of the cam shaft 3 and the cam 60 which is contiguously formed with the cam shaft 3 as shown in FIG. 1.

Here, FIG. 10(a) to FIG. 10(b) show the tappet structural body 6 which mounts the spring seat 10 shown in FIG. 9 thereon. Further, FIG. 11(a) is an upper plan view of the tappet structural body 6 shown in FIG. 10, FIG. 11(b) is a cross-sectional view taken along a line A-A in FIG. 11(a), and FIG. 11(c) is a cross-sectional view taken along a line B-B in FIG. 11(a).

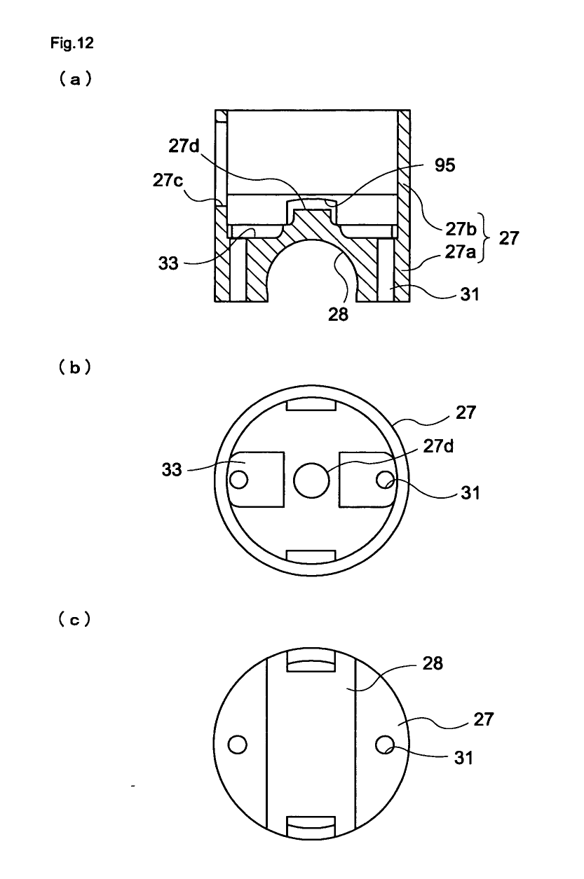

Here, with respect to the tappet body portion which constitutes the tappet structural body, as shown in FIG. 12(a) to FIG. 12(c), it is preferable that the tappet body portion is made of a bearing steel as a whole and is constituted of the body portion 27a made of the block body and the cylindrical slide portion 27b which extends upwardly from an end portion of the body portion 27a. That is, the tappet body portion may preferably be formed in a shape with a circular plane which has an outer peripheral surface which conforms to an inner peripheral surface of the columnar space of the pump housing. Further, in the inside of the cylindrical slide portion 27b, a space in which the spring seat and the plunger are inserted is formed.

Further, as shown in FIG. 12(a), a roller receiver 28 having an inner peripheral surface which conforms to an outer peripheral surface of the roller 29 is formed on the body portion 27a. Further, it is preferable that, by taking diameters, widths and the like of the roller receiver 28 and the roller 29 into consideration, as shown in FIG. 10(b), the roller 29 can be inserted from sides of the roller receiver 28 and the roller 29 is rotatably supported on the roller receiver 28.

Further, as illustrated in FIG. 12(a) to FIG. 12(c), it is preferable to form communicating portions for allowing the lubricant or the lubrication fuel to pass therethrough in the tappet body portion 27. To be more specific, as such communicating portions, it is preferable to form a passing hole 31 in the inside of the tappet body portion 27 and a guide passage 33 at a portion including an upper-surface-side opening portion 31a of the passing hole 31.

The reason is that by forming such passing hole 31 and guide passage 33, it is possible to allow the lubricant or the lubrication fuel to pass between the spring holding chamber and the cam chamber. Accordingly, it is possible to reduce a possibility that the tappet structural body impedes the high-speed driving of the cam and the plunger.

Here, as shown in FIG. 10(b), when the tappet structural body is configured such that the portion of the peripheral portion of the spring seat is extended to restrict the movement of the roller in the rotary axis direction, an insertion hole 95 which allows the insertion of the plate-like restricting means 90a is formed in the tappet body portion 27. Accordingly, by forming a gap 99 around the plate-like restricting means 90a in the insertion hole 95, it is possible to allow the insertion hole 95 to function also as a passing hole through which the lubricant or the like reciprocates.

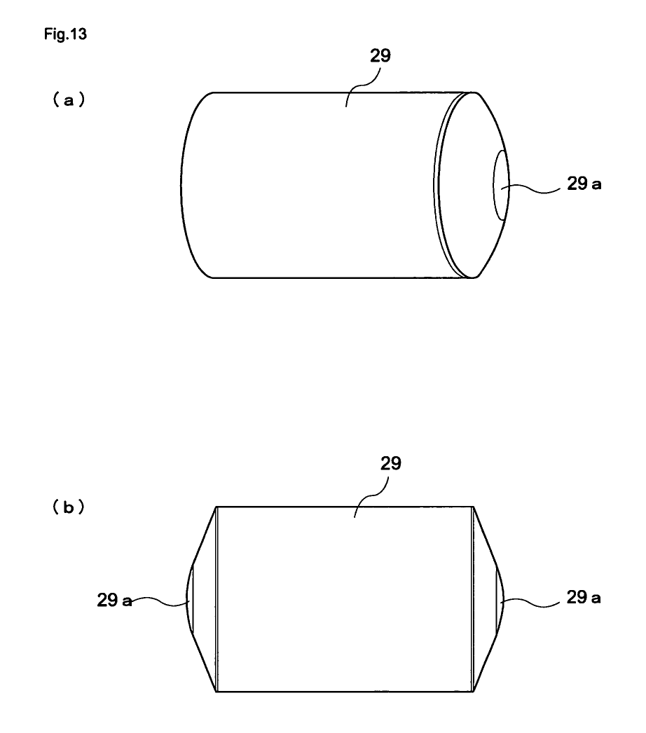

The roller 29 which constitutes the tappet structural body may preferably be, as shown in FIG. 13(a) to FIG. 13(b), configured as an integral body of a pin portion 29a and a roller portion 29b. Further, it is preferable that the roller 29 is inserted into from sideward and is rotatably supported on the roller receiver 28 having a hole surface thereof covered with carbon treatment, for example a carbon coating film.

With the use of the tappet structural body having such constitution, the tappet structural body can reciprocate repeatedly at a high speed for a long period in response to the rotation of the cam which is contiguously connected with the cam shaft.

(8) Cam

The cam 60 is, as illustrated in FIG. 1 and FIG. 2, a main element for converting the rotational movement of the cam 60 into the vertical movement of the plunger 54 by way of the tappet structural body 6. Accordingly, it is preferable that the cam 60 is rotatably inserted and held in the shaft insertion hole 52a by way of the bearing body. Further, the cam 60 is configured to be rotated by driving the cam shaft 3 which is connected with the diesel engine.

On an outer peripheral surface of the cam 60, two cams which are positioned below the columnar spaces 30a, 30b of the pump housing 52 and are arranged in parallel in the axial direction with a predetermined distance are integrally mounted.

(9) Fuel Intake Valve and Fuel Discharge Valve

It is preferable that a fuel intake valve and a fuel discharge valve respectively include a valve body and a valve element which has a flange portion on a distal end thereof and it is preferable that the fuel intake valve 73 and the fuel discharge valve 79 are arranged as shown in FIG. 2.

(10) Fuel Lubrication System

Further, although a lubrication system of the fuel supply pump is not particularly limited, it is preferable to adopt a fuel lubricant system which uses a portion of the fuel oil as a lubrication component (lubricant fuel).

The reason is that with the use of the fuel for the lubrication of the cam chambers and the like, in supplying the fuel into the common rail under pressure by pressurizing the fuel, even when the portion of the fuel for lubricating the cam chamber or the like is mixed into the fuel which is supplied to the common rail under pressure, since these fuels have the same component, there is no possibility that an additive agent or the like which is contained in the lubricant is mixed into the fuel which is supplied to the common rail under pressure as in a case in which the lubricant is used for lubricating the cam chamber or the like. Accordingly, the possibility that the exhaust gas purifying property is lowered can be reduced.

2. Booster-type Accumulator Fuel Injection Device

Further, the fuel supply pump of this embodiment may, for example, preferably be a portion of the booster-type accumulator fuel injection device having the following constitution.

That is, as illustrated in FIG. 14, the booster-type accumulator fuel injection device may preferably be constituted of a fuel tank 102, a feed pump (a low-pressure pump) 104 for supplying the fuel to the fuel tank 102, a fuel supply pump (high-pressure pump) 103, a common rail 106 which constitutes an accumulator for accumulating the fuel supplied from the fuel supply pump 103 under pressure, a booster device (a booster piston) 108 for further pressurizing the fuel which is accumulated by the common rail 106 and a fuel injection device 110.

(1) Fuel Tank, Feed Pump and Fuel Supply Pump

A volume and the configuration of the fuel tank 102 illustrated in FIG. 14 may, for example, preferably be determined by taking into consideration a fact that the fuel supply pump of this embodiment can circulate the fuel at a flow rate per unit time of approximately 500 to 1500 litter/hour.

Further, the feed pump 104 is, as shown in FIG. 14, provided for feeding the fuel (light oil) in the inside of the fuel tank 102 under pressure to the fuel supply pump 103, and a filter 105 may preferably be interposed between the feed pump 104 and the fuel supply pump 103. Further, it is preferable that the feed pump 104, although constituting one example, has the gear pump structure, is mounted on an end portion of a cam and is driven by way of the driving of gears in a state that the feed pump 104 is directly connected with a cam shaft or the feed pump 104 is driven by way of a suitable gear ratio.

Further, it is preferable that the fuel which is fed from the feed pump 104 under pressure by way of the filter 105 is supplied to the fuel supply pump 103 further by way of a proportional control valve 120 which performs an injection quantity control.

Further, it is preferable that the fuel supplied from the feed pump 104 is, in addition to the supply of the fuel under pressure to the proportional control valve 120 and the fuel supply pump 103, made to return to the fuel tank 102 by way of an overflow valve (OFV) which is arranged parallel to the proportional control valve 120. Further, it is preferable that a portion of the fuel is supplied under pressure to the cam chamber of the fuel supply pump 103 by way of an orifice mounted on the overflow valve and is used as the fuel lubricant for the cam chamber.

(2) Common Rail

Further, the constitution of the common rail 106 is not particularly limited and the known constitution may be used. For example, as shown in FIG. 14, it is preferable that a plurality of injectors (injection valves) 110 are connected to the common rail 106, and the fuel which is accumulated at a high pressure in the common rail 106 is injected to the inside of internal combustion engines (not shown in the drawing) from the respective injectors 110.

The reason is that, due to such a constitution, it is possible to inject the fuel into the engine by way of the injector 110 at an injection pressure which conforms to a rotational speed in a state that the injection pressure is not influenced by the fluctuation of the rotational speed of the engine. Further, the conventional injection pump system has a drawback that the injection pressure is changed tracing the engine rotational speed.

Further, a pressure detector 117 is connected to side end of the common rail 106. It is preferable to transmit a pressure detection signal obtained by the pressure detector 117 to an electrical controlling unit (ECU). That is, it is preferable that the ECU, upon receiving the pressure detection signal from the pressure detector 117 controls an electromagnetic control valve (not shown in the drawing) and controls the driving of the proportional control valve in response to the detected pressure.

(3) Booster Device

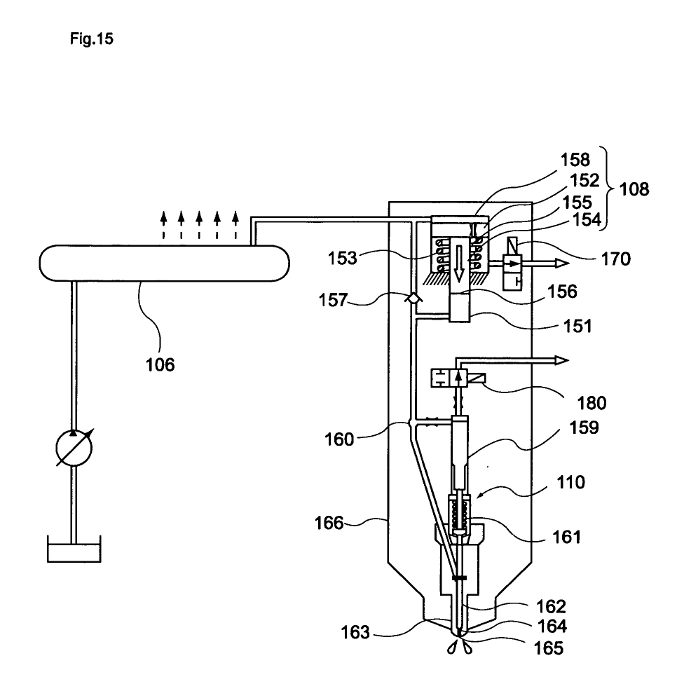

Further, it is preferable that the booster device includes, as illustrated in FIG. 15, a cylinder 155, a mechanical piston (a booster piston) 154, a pressure receiving chamber 158, an electromagnetic valve 170 and a circulation passage 157, wherein the mechanical piston 154 includes a pressure receiving portion 152 having a relatively large area and a pressurizing portion 156 having a relatively small area respectively.

That is, the mechanical piston 154 which is housed in the cylinder 155 is moved by being pushed by the fuel which has the common rail pressure in the pressure receiving portion 152, and the fuel having the common rail pressure of the pressure receiving chamber 158, for example, the pressure of approximately 25 to 100 MPa is further pressurized by the pressurizing portion 156 having the relatively small area thus setting the pressure of the fuel to a value which falls within a range of 150 MPa to 300 MPa.

Further, although a large quantity of fuel having the common rail pressure is used for pressurizing the mechanical piston 154, it is preferable that the fuel is made to return to a fuel inlet of the high-pressure pump by way of an electromagnetic valve 170 after pressurizing. That is, as shown in FIG. 14, it is preferable that the most of fuel having the common rail pressure is, after being used for pressurizing the mechanical piston 154, made to return to the fuel inlet of the high pressure pump 103 by way of a line 121, for example, and the fuel is again used for pressurizing the mechanical piston 154.

On the other hand, the fuel which has the pressure boosted by the pressurizing portion 156 is, as shown in FIG. 15, supplied to the fuel injection device (fuel injection nozzle) 163, is efficiently injected and burnt, and the fuel which flows out from an electromagnetic valve 180 of the fuel injection device is made to return to the fuel tank 102 by way of a line 123.

Accordingly, due to the provision of such a booster device, it is possible to effectively push the mechanical piston under pressure using the fuel having the common rail pressure at an arbitrary timing without excessively increasing the common rail.

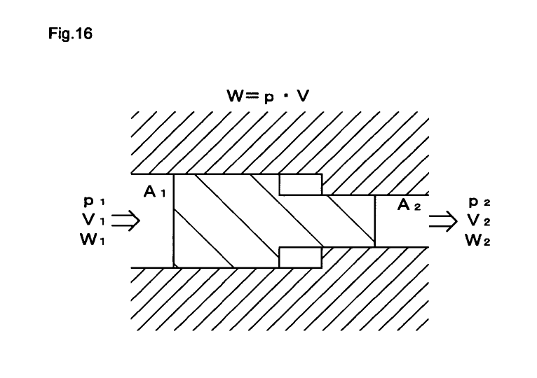

That is, as shown in FIG. 16 which is a schematic view, according to the booster-type accumulator fuel injection device, by providing the pressure receiving portion having the relatively large area and the pressurizing portion having the relatively small area to the mechanical piston and by taking a stroke amount of the mechanical piston into consideration, it is possible to effectively increase the pressure of the fuel having the common rail pressure to a desired value with the least pressurizing loss.

To be more specific, it is possible to receive the fuel by the pressure receiving portion having the relatively large area and to convert the fuel from the common rail (pressure: p1, volume: V1, work load: W1) into the fuel of high pressure (pressure: p2, volume: V2, work load: W2) by the mechanical piston which includes the pressurizing portion having the relatively small area.

(4) Fuel Injection Device

(4)-1 Basic Structure

Further, although the configuration of the fuel injection device (injector) 110 is not particularly limited, for example, as illustrated in FIG. 15, the fuel injection device includes a nozzle body 163 which is constituted of a seat surface 164 on which a needle valve element 162 is seated, and an injection hole 165 which is formed in the nozzle body 163 on the downstream side of a valve-element contact portion of the seat surface 164, wherein when the needle valve element 162 is lifted, the fuel which is supplied from the upstream side of the seat surface 164 is guided to the injection hole 165.

Further, such a fuel injection nozzle 166 may preferably be of an electromagnetic valve type which constantly biases the needle valve element 162 toward the seat surface 164 using a spring 161 or the like and opens or closes the needle valve element 162 in response to the changeover of energization/deenergization of a solenoid 180.

(4)-2 Injection Timing Chart

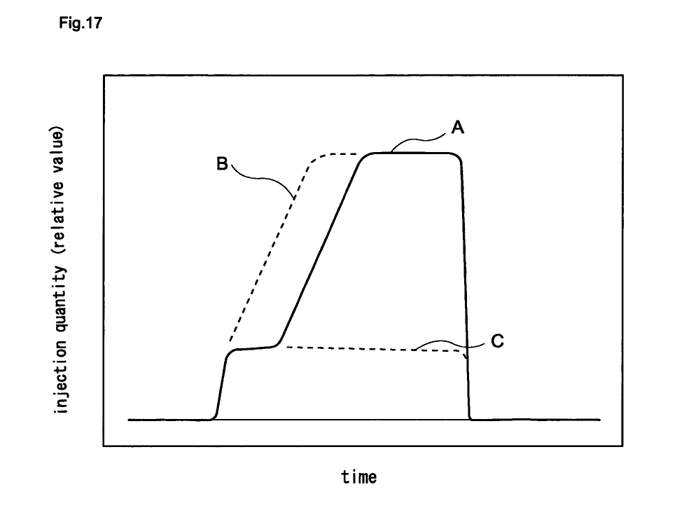

Further, with respect to the injection timing chart of the high-pressure fuel, as illustrated in FIG. 17, it is preferable to adopt a fuel injection chart which possesses the injection state in two stages as indicated by a solid line A.

The reason is that it is possible to obtain the injection timing chart in two stages by combining the common rail pressure and the increased pressure in the booster device (booster piston) and hence, the combustion efficiency of the fuel can be increased and the exhaust gas can be purified.

Further, according to the present invention, it is also preferable to provide the fuel injection timing chart indicated by a dotted line B due to the combination of the common rail pressure and the boosting timing in the booster device (booster piston) as shown in FIG. 17.

Here, when the booster device (booster piston) is not used, that is, in case of the conventional injection timing chart, it is possible to provide the injection timing chart of one stage of low injection quantity as indicated by a dotted line C in FIG. 17.

### INDUSTRIAL APPLICABILITY

According to the fuel supply pump of the present invention, by providing the predetermined inter-cylinder connecting portion, it is possible to allow the lubricant or the lubrication fuel to rapidly and smoothly reciprocate among the plurality of spring holding chambers. Accordingly, even when the pump is rotated at a high speed, there exists a small possibility that the high-speed driving of the plunger is impeded by the lubricant or the like. Accordingly, the fuel supply pump of the present invention can be preferably used as the fuel supply pump which is used in the booster-type accumulator fuel injection device.

Explanation of Symbols

3: cam shaft
6: tappet structural body
10: spring seat
12: spring holding portion
14: plunger mounting portion
27: tappet body portion
27a: body portion
27b: slidable portion
28: roller receiver
29: roller
30: accommodating portion
31: through hole (communicating portion)
33: guide passage
40: inter-cylinder connecting portion
50: fuel supply pump
52: pump housing
53: plunger barrel (cylinder)
54: plunger
60: cam
73: fuel supply valve
74: fuel compression chamber
100: booster type accumulator fuel injection device
102: fuel tank
103: fuel supply pump (high-pressure pump)
104: feed pump (low-pressure pump)
106: common rail
108: piston booster device (booster piston)
110: injector
120: proportion control valves
152: pressure receiving portion
154: mechanical piston
155: cylinder
156: pressure portion
158: pressure receiving portion
166: fuel injection nozzle

1. A fuel supply pump which includes a plurality of plunger barrels, plungers and tappet structural bodies in the inside of a pump housing, wherein,
a plurality of accommodating portions for arranging the plurality of plunger barrels in parallel to each other is formed in the inside of the pump housing and an inter-cylinder connecting portion which allows lubricant or lubricating fuel to pass therethrough is provided between the plurality of accommodating portions.

2. A fuel supply pump according to claim 1, wherein the inter-cylinder connecting portion is arranged at a position higher than an elevated position of the tappet structural body.

3. A fuel supply pump according to claim 1, wherein the inter-cylinder connecting portion is arranged substantially perpendicular to or inclined with respect to the arranging direction of the plurality of plunger barrels.

4. A fuel supply pump according to claim 1, wherein a cross-sectional area of the inter-cylinder connecting portion is set to a value which falls within a range of 10 to 350 mm².

5. A fuel supply pump according to claim 1, wherein a valve portion is provided to a middle portion of the inter-cylinder connecting portion.

6. A fuel supply pump according to claim 1, wherein a communicating portion which allows lubricant or lubricating fuel to pass therethrough is formed in the tappet structural body.

7. A fuel supply pump according to claim 1, wherein a fuel whose flow rate per unit time is 500 to 1500 liter/hour is used in a booster type accumulator fuel injection device which increases a pressure of the fuel to 50 MPa or more.
